(19) United States
(12) Patent Application Publication
(10) Pub. No.: US 2011/0106087 A1
(43) Pub. Date: May 5, 2011

(54) Bone Plate Holder

(76) Inventor: Thomas J. Gamache, Westport, MA (US)

(21) Appl. No.: 12/609,265

(22) Filed: Oct. 30, 2009

(51) Int. Cl. A61B 17/80

(52) U.S. Cl. 606/71; 606/286

(57) ABSTRACT

An instrument for holding a bone plate may include a first arm, a second arm connected to and adjustable relative to the first arm, a first connection tip at the distal end of the first arm configured to connect to the first side wall of the bone plate, and a second connection tip at the distal end of the second arm configured to connect to the second side wall of the bone plate. The first connection tip may have a planar distal surface and the second connection tip may have a planar distal surface. The planar distal surface of the first connection tip, the planar distal surface of the second connection tip, and the distal bone engaging surface of the bone plate may be co-planar when the first connection tip and the second connection tip connect to the first side wall and the second side wall, respectively.

### BACKGROUND

In many spinal and orthopedic procedures a bone plate is used to fix bone segments, such as adjacent vertebrae. In laminoplasty procedures, for example, in which the lamina of a vertebra is cut and spaced apart to expand the spinal canal, a laminoplasty plate is frequently used to stabilize the cut vertebra after expansion of the spinal canal. Manipulating the laminoplasty plate into the proper position and anchoring it to the vertebra can be challenging, as laminoplasty plates are small and difficult to handle. For this reason, there is a need for improved instruments and techniques for manipulating laminoplasty plates and other types of bone plates.

### SUMMARY

Disclosed herein are bone plate systems including a plurality of bone plates of differing lengths, such as a laminoplasty system having a plurality of laminoplasty plates of differing lengths, and an instrument for holding one of the bone plates. The instrument is particularly suited for manipulating bone plates into position relative to one or more bone segments and maintaining the bone plate in position as the bone plate is anchored to the bone segment.

In one aspect, an instrument for holding a bone plate may include a first arm having a proximal end and a distal end, a second arm connected to and adjustable relative to the first arm, a first connection tip at the distal end of the first arm configured to connect to the first side wall of the bone plate, and a second connection tip at the distal end of the second arm configured to connect to the second side wall of the bone plate. The first connection tip may have a planar distal surface and the second connection tip may have a planar distal surface. The planar distal surface of the first connection tip, the planar distal surface of the second connection tip, and the distal bone engaging surface of the bone plate may be co-planar when the first connection tip and the second connection tip connect to the first side wall of the bone plate and the second side wall of the bone plate, respectively.

### BRIEF DESCRIPTION OF THE FIGURES

These and other features and advantages of the devices and methods disclosed herein will be more fully understood by reference to the following detailed description in conjunction with the attached drawings in which like reference numerals refer to like elements through the different views. The drawings illustrate principles of the devices and methods disclosed herein and, although not to scale, show relative dimensions.

FIG. 1 is a perspective view of a laminoplasty system including a plurality of bone plates,

FIG. 2 is a side view of a bone plate of the laminoplasty system of FIG. 1,

FIG. 3 is a top view of the bone plate of FIG. 2,

FIG. 4 is a perspective view of the bone plate of FIG. 2,

FIG. 5 is a front view of the bone plate of FIG. 2,

FIG. 6 is a side view of a bone plate and a bone graft of the laminoplasty of FIG. 1, illustrating the bone graft connected to the bone plate,

FIG. 7 is a perspective view of an instrument for holding a bone plate, illustrating the instrument holding a bone plate,

FIG. 8 is a side view of the instrument of FIG. 7,

FIG. 9 is a front view of the instrument of FIG. 7,

FIG. 10 is a top view of the instrument of FIG. 7,

FIG. 11 is a cross sectional view of the connection tips of the instrument of FIG. 7, illustrating the connections tips without a bone plate positioned therebetween,

FIG. 12 is a perspective view of the distal end of the instrument of FIG. 7, illustrating the instrument connected to a bone plate in an alternate orientation,

FIG. 13 is a side view of the distal end of the instrument of FIG. 12,

FIG. 14 is a cross sectional view of the distal end of the instrument of FIG. 12, and

FIGS. 15 and 16 are perspective views of a laminoplasty procedure, illustrating a bone plate being positioned against a vertebra by the instrument of FIG. 7.

### DETAIL DESCRIPTION OF EXEMPLARY EMBODIMENTS

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

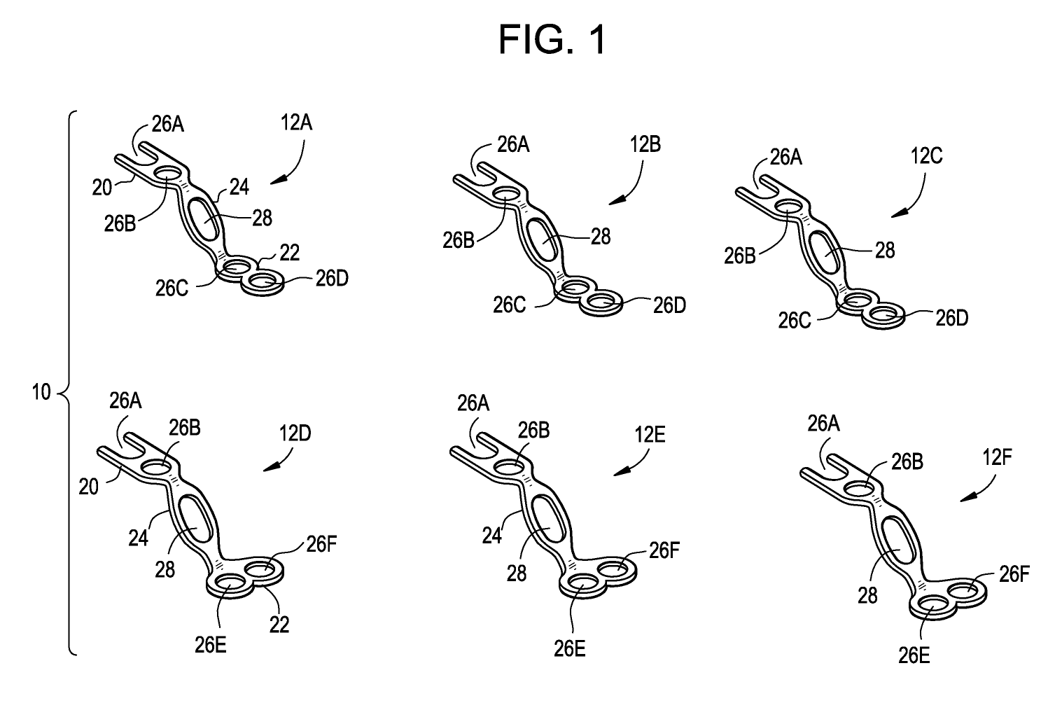

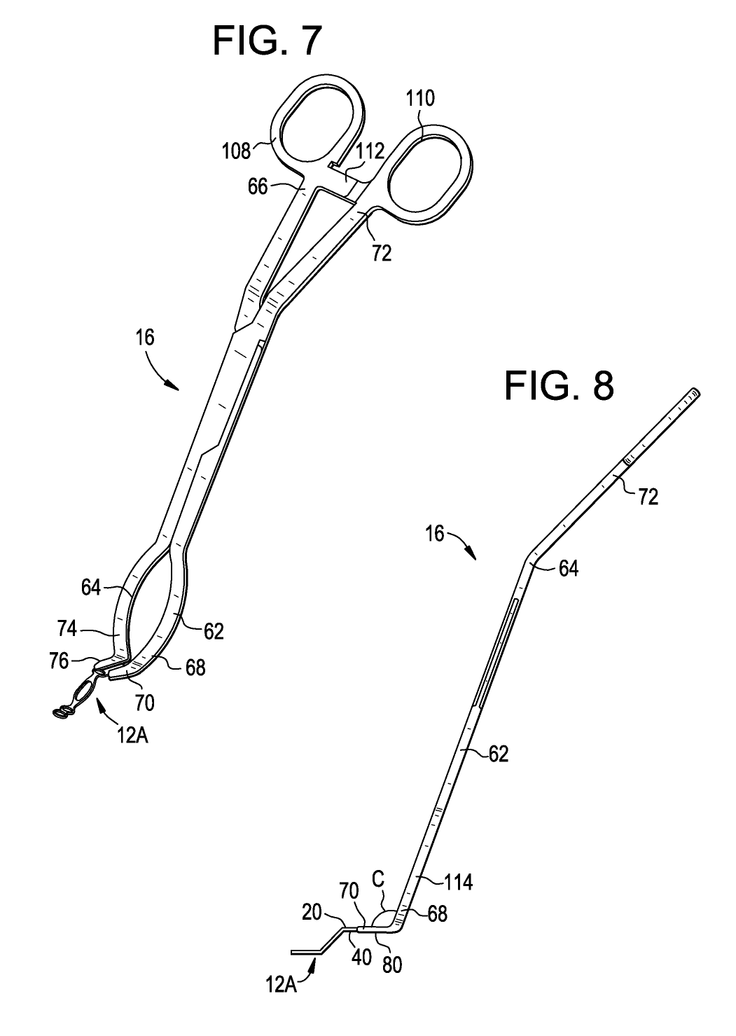

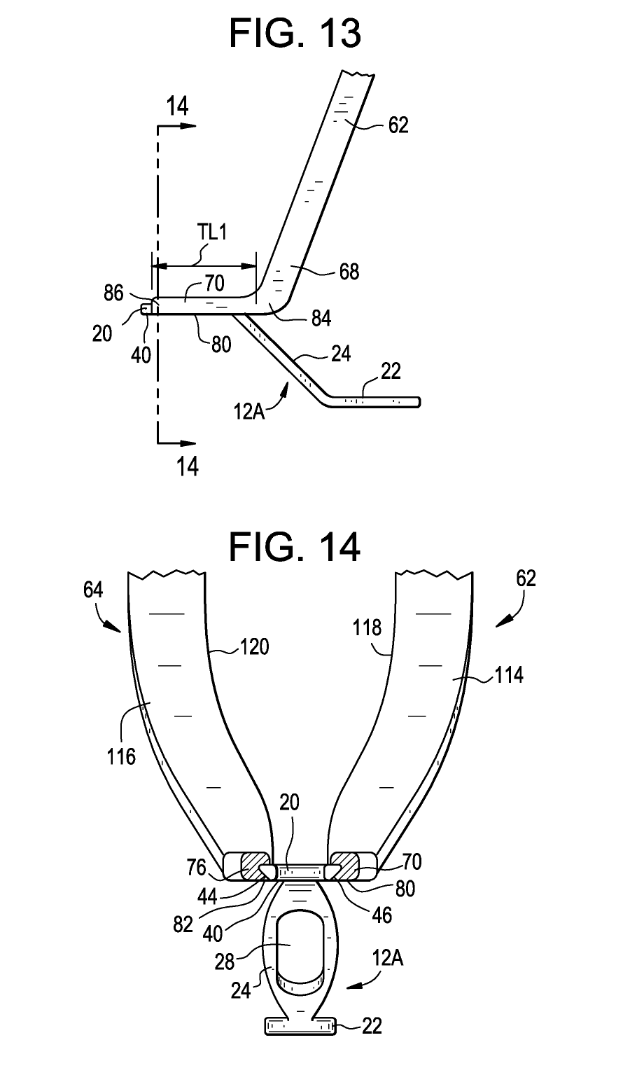

FIG. 1 illustrates an exemplary laminoplasty system 10 including a set of bone plates 12. The exemplary bone plates 12 are particularly suited for use in a surgical procedure to alleviate spinal cord compression by expanding the spinal canal, typically by displacing bone, such as the lamina, that surrounds the spinal cord. This procedure, generally referred to as laminoplasty, typically involves making two types of cuts into the lamina, a complete osteotomy through the lamina on one side of the spine and a partial osteotomy, or “green stick”, on the contra-lateral lamina, which preserves the bottom cortex and creates a flexible hinge in the bone. This bridge of lamina bone is then levered open about the hinge and fixed with one of the plates 12 and a plurality of screws. In some procedures, a bone graft may be connected to the bone plate and positioned in the expanded lamina, typically between the cut lamina and the lateral mass of the vertebra, to facilitate fusion of the lamina to the lateral mass. The exemplary laminoplasty system 10, thus, also may include a plurality of bone grafts of differing length. Exemplary laminoplasty plates, bone grafts, and methods of expanding the spinal canal of a vertebra are described in commonly owned, co-pending U.S. patent application Ser. No. ______, entitled “Laminoplasty Plates and Methods of Expanding the Spinal Canal” (Attorney Docket No. DEP6267USNP) concurrently filed herewith, which is incorporated herein by reference. Typically, a bone graft 14 is connected to the bone plate 12 prior to connecting the plate 12 to the vertebra, as illustrated in FIG. 6. The exemplary laminoplasty system also includes an instrument 16 for holding one of the bone plates, and if a bone graft is employed, the bone graft connected to the bone plate, as illustrated in FIGS. 7-16.

The set of bone plates 14 may include a plurality of bone plates of varying sizes and shapes. For example, a set of bone plates 14 may include a plurality of bone plates of two types: in-line bone plates 12A-C and offset bone plates 12D-F, as illustrated in FIG. 1. The exemplary in-line plates 12A-C include a plurality of bone screw receiving holes aligned along the central longitudinal axis CA of the bone plate. The exemplary off-set plates 12D-F include a plurality of bone screw receiving holes, at least two of which are off-set from the central longitudinal axis of the bone plate. The exemplary in-line bone plates 12A-C and offset bone plates 12D-F may come in differing lengths in the system 10. During the surgical procedure, the surgeon may select an appropriate length bone plate based on the length of the space between bone segments the bone plate is to connect. The bone plates 12 may be constructed of any biocompatible material suitable for implantation within the human body, including, for example, stainless steel, titanium and titanium alloys, and polymers, including biodegradable polymers. In one exemplary laminoplasty system, the inline bone plates and the offset bone plates are provided in the following differing lengths: 6 mm, 8 mm, 10 mm, 12 mm, 14 mm, 16 mm, and 18 mm.

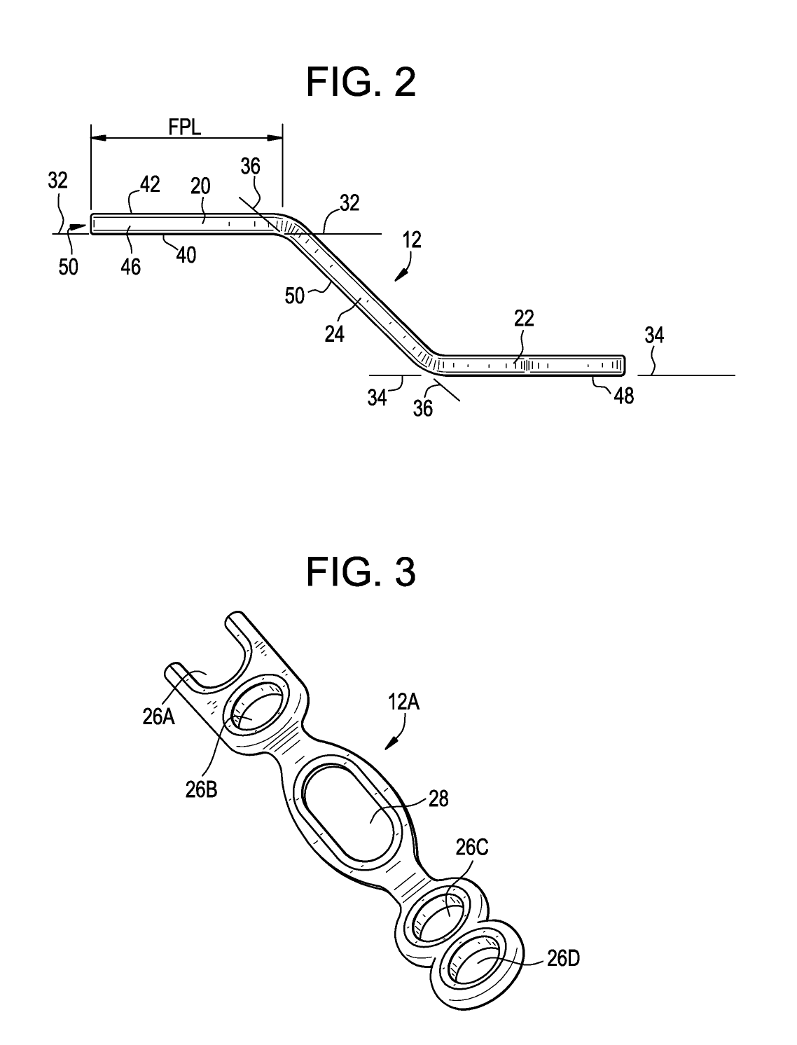

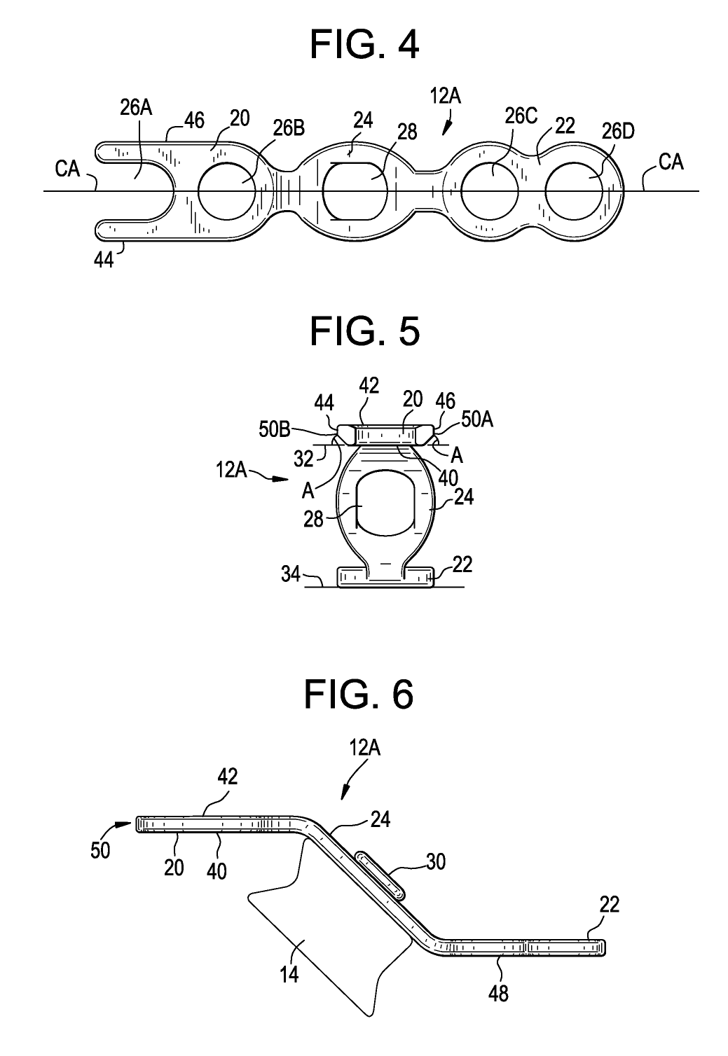

Referring to FIGS. 2-6, the exemplary bone plates 12, including bone plates 12A-12D, may include a first plate segment for connecting to a first bone segment, such as the lamina of a vertebra, a second plate segment for connecting to a second bone segment, such as the lateral mass of the vertebra, and a central plate segment for holding a bone graft positioned between the first and second bone segments if a bone graft is employed. Referring to FIGS. 2-4, for example, exemplary in-line bone plate 12A includes a first plate segment 20, a second plate segment 22, and a central plate segment 24 interposed between the first plate segment 20 and the second plate segment 22. The first plate segment 20 may include one or more bone anchor receiving holes. In the exemplary embodiment, for example, the first plate segment 20 includes a first bone anchor receiving hole 26A in the form of an open ended slot and an enclosed, generally circular second bone anchor receiving hole 26B. The second plate segment 22 may include one or more bone anchor receiving holes. In the exemplary embodiment, for example, the second plate segment 22 includes a third bone anchor receiving hole 26C and a fourth bone anchor receiving hole 26D. In the in-line bone plates 12A-C, the first, second, third and fourth bone anchor receiving holes 26A-D are aligned along the central longitudinal axis CA of the bone plate. The central plate segment 24 may include one or more graft openings for receiving an anchor 30 to connect to one of the bone grafts 14. In the exemplary bone plate 12A, for example, the central plate segment 24 includes an elongated, closed slot 28.

The off-set plates 12D-12F of the system 10 may have configuration analogous to that of the in-line plates 12A-12C except the second segment of the off-set plates includes two off-set bone anchor receiving holes 26E, 26F that are positioned off-set from the central longitudinal axis of the bone plate.

The first plate segment 20 includes a planar, distal first bone engaging surface 40, a proximal surface 42, and a first side wall 44 and a second side wall 46 connected by the distal first bone engaging surface 40 and by the proximal surface 42. The second plate segment 22 includes a distal second bone engaging surface 48 and the central plate segment includes a distal third surface 50. The bone plates 12 of the exemplary system 10 may have a geometry to facilitate connection to the lamina of a vertebra at one end, e.g., at the first plate segment, and connection to the lateral mass of the vertebra at the other end, e.g. at the second plate segment. Referring to FIG. 2, for example, the first bone engaging surface 40 of the first plate segment 20 and the second bone engaging surface 48 of the second plate segment 22 of the exemplary bone plate 12 lie in separate, approximately parallel planes, e.g., first plane 32 and second plane 34, respectively, and the distal third surface 50 of the central plate segment 24 connects the first bone engaging surface 40 and the second bone engaging surface 48 and lies in a plane, e.g., third plane 36 that intersects the parallel planes 32 and 34. The first plate segment 20 includes a first plate length FPL extending from a first end 50 to the junction with the central plate segment 24. The first side wall 44 and the second side wall 46 of the exemplary plate 12A each include an angled section 50A, 50B extending from the first distal bone engaging surface 40 toward the proximal surface 42 of the first plate segment 20. In the exemplary plate 12A, the angled sections 50A, 50B are angled away from each other and are oriented at an angle A relative to the first plane 32 that is less than 90°.

A bone graft 14 may be connected to the laminoplasty plate 12A using an instrument that holds the bone graft 14 relative to the plate during insertion of the anchor 30. An exemplary instrument is described in commonly owned, co-pending U.S. patent application No. ______, entitled “Bone Graft Loading Instruments and Methods of Connecting a Bone Graft to a Bone Plate” (Attorney Docket No. DEP6268USNP) concurrently filed herewith, which is incorporated herein by reference.

Referring to FIG. 7-15, an exemplary instrument 16 for holding one of the plurality of bone plates 12 includes a first arm 62 and a second arm 64 connected to and adjustable relative to the first arm 62. The first arm 62 has a proximal end 66 and a distal end 68. The exemplary instrument 16 includes a first connection tip 70 at the distal end 68 of the first arm 62. The second arm 64 has a proximal end 72 and a distal end 74 and the exemplary instrument 16 includes a second connection tip 76 at the distal end 74 of the second arm 64. The first connection tip 70 of the instrument 16 is configured to connect to one of the side walls of the first plate segment 20 of one of the bone plates 12 of the system 10, e.g., the first side wall 44 or the second side wall 46 of the first plate segment 12A. The second connection tip 76 configured to connect to the other side wall of the first plate segment 20 of the bone plate, e.g., the first side wall 44 or the second side wall 46. The first arm 62 and the second arm 64 being adjustable between a space apart position in which the first connection tip 70 and the second connection tip 76 are spaced apart from each other to facilitate placement of the first plate segment 20 between the first connection tip 70 and the second connection tip 76 and an engaged position in which the first connection tip 70 and the second tip 76 are connected to the first plate segment 20.

The first connection tip 70 and the second connection tip 76 may have a geometry to facilitate placement of a bone plate directly against a bone segment. In the exemplary instrument 16, for example, the first connection tip 70 has a planar distal surface 80 and the second connection tip 76 has a planar distal surface 82. The planar distal surface 80 of the first connection tip 70, the planar distal surface 82 of the second connection tip 76, and the first bone engaging surface 40 of the first plate segment 20 are co-planar when the first connection tip 70 and the second connection tip 76 are connected to side walls of the first plate segment 20. This co-planar configuration allows the first bone engaging surface 40 to be placed directly against a bone segment, e.g., a vertebra, without a portion of the instrument interposed between the plate and the bone and, in laminoplasty procedures, without a portion of the instrument extending into the spinal canal.

The first connection tip 70 and the second connection tip 76 may have length sufficient to engage a sufficient length of the side walls of a bone plate to provide a stable connection during manipulation and anchoring of the bone plate. For example, the first connection tip 70 of the exemplary instrument may extend from a first junction 84 with the distal end 68 of the first arm 62 to a free end 86 of the first connection tip 70. The first connection tip 70 may have a first tip length TL1 extending from the free end 86 to the first junction 84. The second connection tip 76 may extend from a second junction 88 with the distal end 74 of second arm 64 to a free end 90 and the second connection tip 76 may have a second tip length TL2 extending from the free end 90 to the second junction 88. In the exemplary instrument 16, the first tip length TL1 may be approximately equal to the second tip length TL2 and the first tip length TL1 and the second tip length TL2 may be greater than or approximately equal to the first plate segment length FPL. In alternative embodiments, the first tip length TL1 and the second tip length TL2 may have length less than the length of the first plate length or the plate length of the selected bone plate.

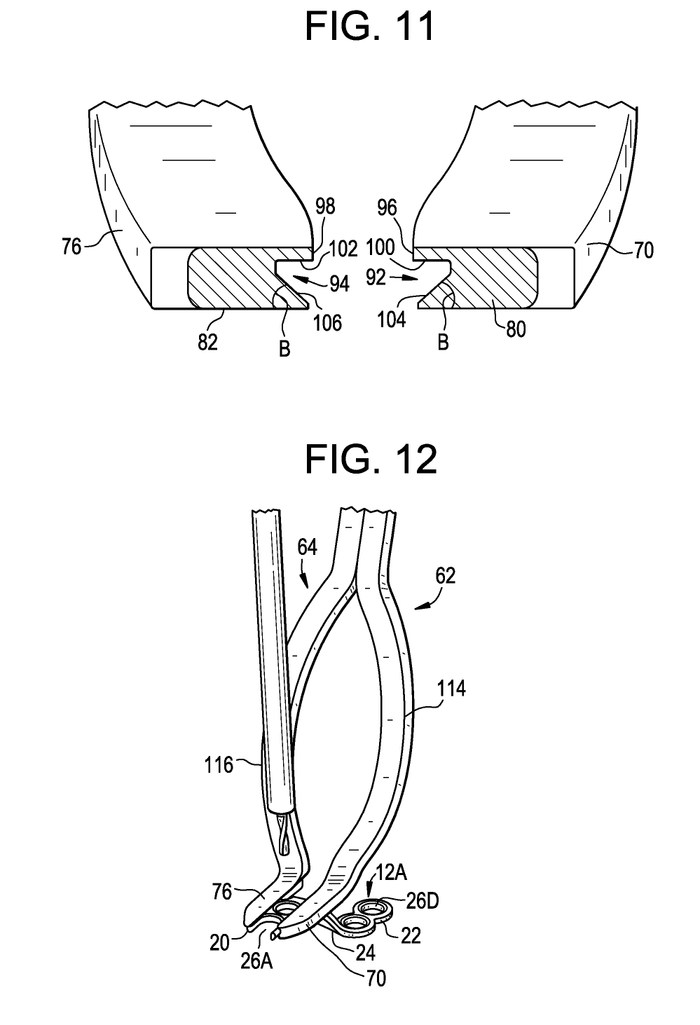

Referring to FIGS. 11 and 14, the first connection tip 70 and/or the second connection tip 76 may have a feature to facilitate connection of the connection tips to the selected bone plate. In the exemplary embodiment, for example, the first connection tip 70 has a first groove 92 for receiving the first side wall 44 (or second wall 46) of a bone plate 12A upon connection with the first plate segment 20 of the bone plate 12A. The first groove 92 may extend along at least a portion of the first tip length TL1 and, in the exemplary instrument 16, may extend along the entirety of the first tip length TL1 from the free end 86 to the first junction 84. The second connection tip 76 may have a second groove 94 for receiving the second side wall 46 (or the first side wall 44) upon connection of the second connection tip 76 with the first plate segment 20. The second groove 94 may extend along at least a portion of the second tip length TL2 and, in the exemplary instrument 16, may extend along the entirety of the second tip length TL2 from the free end 90 to the first junction 88. The first groove 92 may be formed in a first inner side wall 96 of the first connection tip 70 and may be open along the first inner side wall 96 to receive the first side wall 44 of the first plate segment 20. The second groove 94 may be formed in a second inner side wall 98 of the second connection tip 76 and may be open along the second inner side wall 98 to receive the second side wall 46 of the first plate segment 20. As the first inner side wall 96 and the second inner wall 98 face each other when engaging a bone plate, the opening in the first groove 92 and the opening in the second groove 94 face each other.

The first groove 92 and the second groove 94 may have a shape that is complementary to the shape of the bone plate received by the grooves. In the exemplary instrument, for example, the first groove 92 has a shape that is complementary in shape to the first and second walls 44, 46 of the first plate segment 20. The first groove 92 may have a distal facing, planar top surface 100 for engaging the proximal surface 42 of the first plate segment 20 and the second groove 94 has a distal facing, planar top surface 102 for engaging the proximal surface 42 of the first plate segment 20. The first groove 92 may have an angled side wall 104 that extends from the planar distal surface 80 of the first connection tip 72 and is oriented at an angle B relative to the distal bone engaging surface 80. The second groove 94 may have an angled side wall 106 that extends from the planar distal surface 82 of the second connection tip 76 and is oriented at the angle B relative to the distal bone engaging surface 82. In the exemplary instrument 16, the angle B is approximately equal to the angle A of the angled sections 50A, 50B of the first and second side walls 44, 46, respectively.

Continuing to refer to FIGS. 7-15, the first arm 62 is pivotally connected to the second arm 64 to provide for pivotal adjustment of the first arm 62 and the second arm 64 the spaced apart position and the engaged position. The proximal end 66 of the first arm 62 and the proximal end 72 of the second arm 64 each may include a handle 108, 110 or the like to facilitate adjustment of the arms. The instrument 16 may also include a locking mechanism, such as ratchet 112, to lock the position of the first arm 62 and the second arm 64.

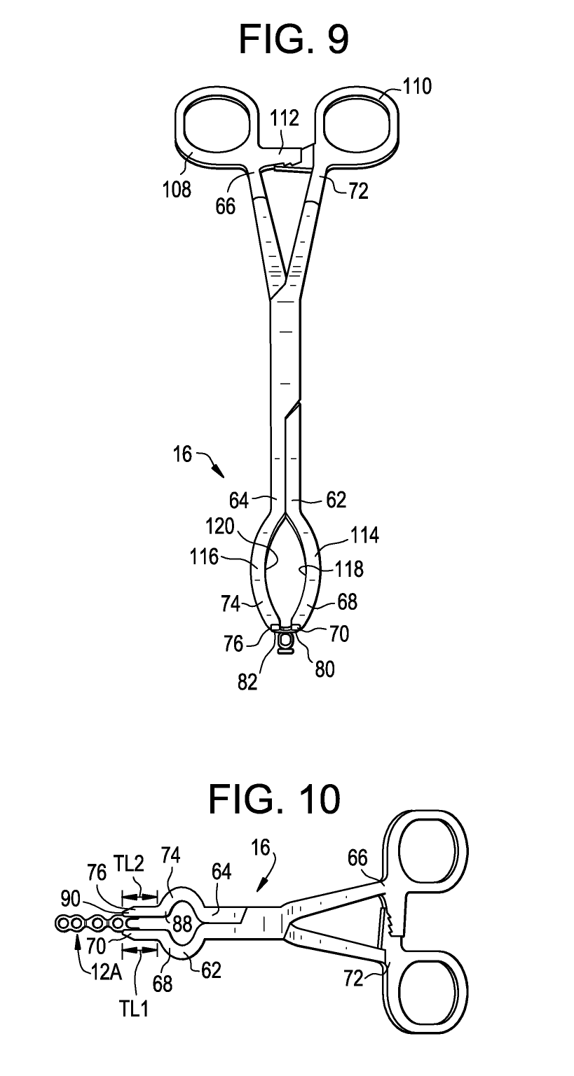

The first arm 62 and the second arm 64 of the exemplary instrument may be configured to facilitate placement of additional instruments, such as a drill 150 or a screwdriver, and implants, such as bone anchors, to the bone plate connected to the connection tips 70, 76 of the instrument. Referring to FIG. 9, for example, the first arm 62 may include an arcuate segment 114 extending proximally from the first connection tip 70 and a second arcuate segment 116 extending proximally from the second distal connection tip 76. The first arcuate segment 114 includes a first concave inner side wall 118 and the second arcuate segment 116 includes a second concave inner side wall 120 that faces the first concave inner side wall 118 and thereby provides increased space between the first arm 62 and the second arm 64 to access the bone plate connected to the instrument 16 and to provide increased visibility of the anatomy in proximity to the bone plate. The first arcuate segment 114 may be oriented at an angle C of greater than 90° relative to the first connection tip 70 and the second arcuate segment 114 may be oriented at an angle greater than 90° relative to the second connection tip 76, as illustrated in FIG. 8.

In use, the exemplary instrument 16 may be connected to the first plate segment 20 of the exemplary bone plate 12A by engaging the first connection tip 70 to one of the sidewall of the first plate segment 20 and by engaging the second connection tip 76 to the other sidewall of the first plate segment 20. The first plate segment 20 may be connected to the instrument 16 in one of two orientations: (1) first connection tip 70 engaging first side wall 44 and the second connection tip 76 engaging the second side wall 46, as illustrated in FIGS. 7-10 and 15-16, and (2) first connection tip 70 engaging second side wall 46 and the second connection tip 76 engaging the first side wall 44, as illustrated in FIGS. 12-14. In both orientations, the planar distal surface 80 of the first connection tip 70, the planar distal surface 82 of the second connection tip 76, and the first bone engaging surface 40 of the first plate segment 20 are co-planar when the first connection tip 70 and the second connection tip 76 are connected to side walls of the first plate segment 20.

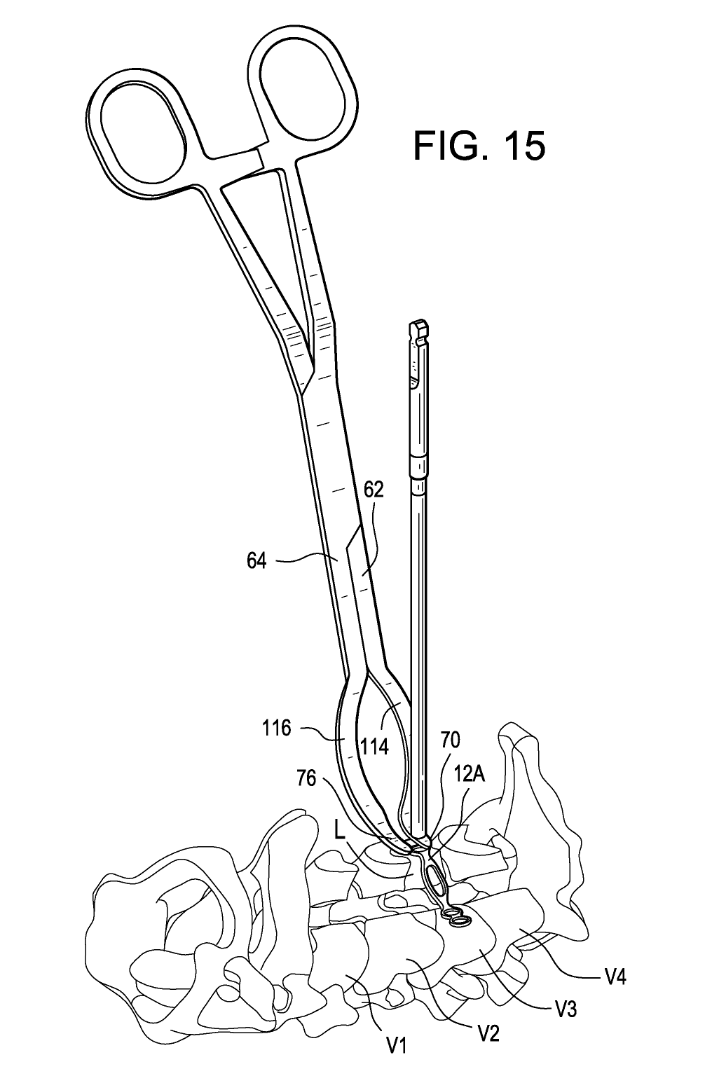

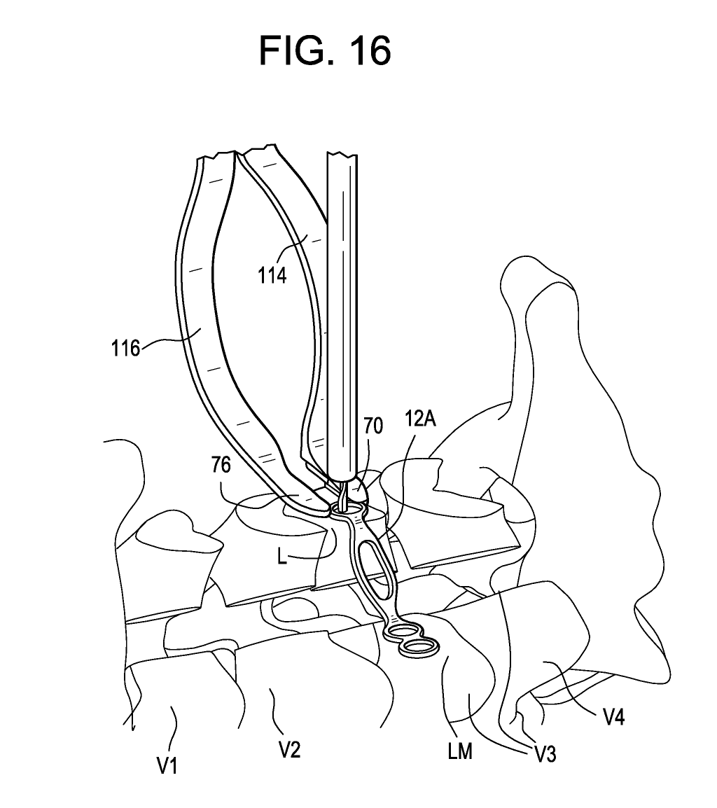

In a laminoplasty procedure, the instrument 16 may be used to manipulate a bone plate, such as plate 12A, into position relative to a vertebra and hold the plate in position while bone anchors are implanted to secure the plate to the vertebra. FIGS. 15 and 16 illustrate an open door laminoplasty procedure to alleviate spinal cord compression by expanding the spinal canal by displacing the lamina of multiple vertebrae that surround the spinal cord. In the illustrated procedure, the lamina of a plurality of adjacent vertebra V1-V4 are completely cut on one side of the spine and on the contra-lateral lamina of each vertebra, a partial cut of the other lamina is made to create a flexible hinge in the bone. This bridge of lamina bone is then levered open about the hinge to expand the spinal canal. Bone plate 12A is manipulated using the instrument 16 to position the first plate segment 20 against the lamina L of the vertebra V3 and to position the second plate segment 22 against the lateral mass LM of the vertebra V3. Once positioned, a drill 150 may be used to drill holes in the lamina and lateral mass for receiving bone anchors. With the instrument 16 holding the bone plate 12A in position, the drill may be positioned through the bone anchor receiving holes 26A-D to drill the requisite holes. With the instrument 16 continuing to hold the bone plate 12A in position, bone anchors may then be positioned through the bone anchor receiving holes 26A-D to anchor the bone plate 12A. Instrument 16 may then be disconnected from the first plate segment 20 by separating the first connection tip 70 and the second connection tip 76.

While the present instrument 16 has been described in connection with exemplary laminoplasty plate 12A and the other laminoplasty plates of the system 10, the instrument 16 may be used with any type of bone plate, including, for example, anterior cervical plates, anterior lumbar plates, trauma plates, and fracture fixation plates.

While the devices and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A laminoplasty system comprising:
a set of bone plates, a plurality of the bone plates of the set of bone plates including a first plate segment, a second plate segment, and a central plate segment interposed between the first plate segment and the second plate segment, the first plate segment including a first hole for receiving a bone anchor, the second plate segment including a second hole for receiving a bone anchor, the central plate segment including a graft opening for receiving an anchor to connect one of the bone grafts, the first plate segment including a planar, distal first bone engaging surface, a proximal surface, and a first side wall and a second side wall connected by the distal first bone engaging surface and by the proximal surface, the second plate segment including a distal second bone engaging surface, the central plate segment including a distal third surface, the first bone engaging surface and the second bone engaging surface lying in separate approximately parallel planes and the distal third surface connecting the first bone engaging surface and the second bone engaging surface and lying in a plane that intersects the parallel planes, and
an instrument for holding one of the plurality of bone plates, the instrument comprising a first arm and a second arm connected to and adjustable relative to the first arm, the first arm having a proximal end and a distal end, the second arm having a proximal end and a distal end, the instrument having a first connection tip at the distal end of the first arm and a second connection tip at the distal end of the second arm, the first connection tip configured to connect to the first side wall of the first plate segment, the second connection tip configured to connect to the second side wall of the first plate segment, the first connection tip having a planar distal surface, the second connection tip having a planar distal surface, the planar distal surface of the first connection tip, the planar distal surface of the second connection tip, and the first bone engaging surface being co-planar when the first connection tip and the second connection tip connect to the first side wall and the second side wall, respectively.

2. The system of claim 1, wherein the first connection tip extends from a first junction with the distal end of the first arm to a free end, the first connection tip having a first tip length extending from the free end to the first junction and wherein the second connection tip extends from a second junction with the distal end of second arm to a free end, the second connection tip having a second tip length extending from the free end to the second junction, the first tip length being equal to the second tip length.

3. The system of claim 2, wherein the first plate segment has a first plate segment length and the first tip length and the second tip length are greater than or equal to the first plate segment length.

4. The system of claim 2, wherein the first connection tip has a first groove for receiving the first side wall upon connection with the first plate segment, the first groove extending along the first tip length from the free end to the first junction and wherein the second connection tip has a second groove for receiving the second side wall upon connection with the first plate segment, the second groove extending along the second tip length from the free end to the second junction.

5. The system of claim 4, wherein the first connection tip has a first inner side wall facing a second inner side wall of the second connection tip, the first groove opening along the first inner side wall to receive the first side wall of the first plate segment, and wherein the second groove opens along the second inner side wall to receive the second side wall of the first plate segment.

6. The system of claim 5, wherein the first groove has a distal facing, planar top surface for engaging the proximal surface of the first plate segment and the second groove has a distal facing, planar top surface for engaging the proximal surface of the first plate segment.

7. The system of claim 6, wherein the first groove has an angled side wall that extends from the planar distal surface of the first connection tip and the second groove has an angled side wall that extends from the planar distal surface of the second connection tip.

8. The system of claim 1, wherein the first arm is pivotally connected to the second arm, first arm and the second arm pivoting between a space apart position in which the first connection tip and the second connection tip are spaced apart from each other to facilitate placement of the first plate segment between the first connection tip and the second connection tip and an engaged position in which the first connection tip and the second tip are connected to the first plate segment.

9. The system of claim 1, wherein the first arm includes a first arcuate segment extending proximally from the first connection tip and the second arm includes a second arcuate segment extending proximally from the second connection tip.

10. The system of claim 9, wherein the first arcuate segment includes a first concave inner side wall and the second arcuate segment includes a second concave inner side wall that faces the first concave inner side wall.

11. The system of claim 9, wherein the first arcuate segment is oriented at an angle of greater than 90° relative to the first connection tip and the second arcuate segment is oriented at an angle greater than 90° relative to the second connection tip.

12. An instrument for holding a bone plate having a distal bone engaging surface, a proximal surface, and a first side wall and a second side wall connected by the distal first bone engaging surface and by the proximal surface, the instrument comprising:
a first arm having a proximal end and a distal end,
a second arm connected to and adjustable relative to the first arm, the second arm having a proximal end and a distal end,
a first connection tip at the distal end of the first arm, the first connection tip configured to connect to the first side wall of the bone plate, and
a second connection tip at the distal end of the second arm, the second connection tip configured to connect to the second side wall of the bone plate, the first connection tip having a planar distal surface, the second connection tip having a planar distal surface, the planar distal surface of the first connection tip, the planar distal surface of the second connection tip, and the distal bone engaging surface of the bone plate being co-planar when the first connection tip and the second connection tip connect to the first side wall and the second side wall, respectively.

13. The instrument of claim 12, wherein the first connection tip extends from a first junction with the distal end of the first arm to a free end, the first connection tip having a first tip length extending from the free end to the first junction and wherein the second connection tip extends from a second junction with the distal end of second arm to a free end, the second connection tip having a second tip length extending from the free end to the second junction, the first tip length being equal to the second tip length.

14. The instrument of claim 13, wherein the bone plate has a plate length and the first tip length and the second tip length are greater than or equal to the plate length.

15. The instrument of claim 13, wherein the first connection tip has a first groove for receiving the first side wall upon connection with the bone plate, the first groove extending along the first tip length from the free end to the first junction and wherein the second connection tip has a second groove for receiving the second side wall upon connection with the bone plate, the second groove extending along the second tip length from the free end to the second junction.

16. The instrument of claim 15, wherein the first connection tip has a first inner side wall facing a second inner side wall of the second connection tip, the first groove opening along the first inner side wall to receive the first side wall of the bone plate, and wherein the second groove opens along the second inner side wall to receive the second side wall of the bone plate.

17. The instrument of claim 16, wherein the first groove has a distal facing, planar top surface for engaging the proximal surface of the bone plate and the second groove has a distal facing, planar top surface for engaging the proximal surface of the bone plate.

18. The instrument of claim 17, wherein the first groove has an angled side wall that extends from the planar distal surface of the first connection tip and the second groove has an angled side wall that extends from the planar distal surface of the second connection tip.

19. The instrument of claim 12, wherein the first arm is pivotally connected to the second arm, first arm and the second arm pivoting between a space apart position in which the first connection tip and the second connection tip are spaced apart from each other to facilitate placement of the bone plate between the first connection tip and the second connection tip and an engaged position in which the first connection tip and the second tip are connected to the bone plate.

20. The instrument of claim 12, wherein the first arm includes a first arcuate segment extending proximally from the first connection tip and the second arm includes a second arcuate segment extending proximally from the second connection tip.

21. The instrument of claim 20, wherein the first arcuate segment includes a first concave inner side wall and the second arcuate segment includes a second concave inner side wall that faces the first concave inner side wall.

22. The instrument of claim 20, wherein the first arcuate segment is oriented at an angle of greater than 90° relative to the first connection tip and the second arcuate segment is oriented at an angle greater than 90° relative to the second connection tip.

23. A bone plate system comprising:
a set of bone plates of differing lengths, a plurality of the bone plates of the set of bone plates including a planar, distal first bone engaging surface, a proximal surface opposite to the distal bone engaging surface, and a first side wall and a second side wall connected by the distal first bone engaging surface and by the proximal surface, and
an instrument for holding one of the plurality of bone plates, the instrument comprising a first arm and a second arm connected to and adjustable relative to the first arm, the first arm having a proximal end and a distal end, the second arm having a proximal end and a distal end, the instrument having a first connection tip at the distal end of the first arm and a second connection tip at the distal end of the second arm, the first connection tip configured to connect to the first side wall of a first one of the plurality of bone plates, the second connection tip configured to connect to the second side wall of the first bone plate, the first connection tip having a planar distal surface, the second connection tip having a planar distal surface, the planar distal surface of the first connection tip, the planar distal surface of the second connection tip, and the distal bone engaging surface of the first bone plate being co-planar when the first connection tip and the second connection tip connect to the first side wall and the second side wall, respectively.
